(19) United States
(12) Patent Application Publication
(10) Pub. No.: US 2025/0269033 A1
(43) Pub. Date: Aug. 28, 2025

(54) A COMPOSITION COMPRISING A THERMORESPONSIVE POLYMER AND A PHARMACEUTICAL FORMULATION COMPRISING THE SAME

(71) Applicant: Nanoform Finland Oyj, Helsinki (FI)

(72) Inventors: Tamas SOLYMOSI, Helsinki (FI); Panu NOPPARI, Helsinki (FI); Satu LAKIO, Helsinki (FI)

(21) Appl. No.: 18/857,969

(22) PCT Filed: Apr. 4, 2023
(86) PCT No.: PCT/FI2023/050184

§ 371 Date: Oct. 18, 2024

(30) Foreign Application Priority Data
Apr. 21, 2022 (FI) 20225332

(51) Int. Cl. A61K 47/32; A61K 47/12; A61K 47/26

(52) U.S. Cl. CPC A61K 47/32; A61K 47/12; A61K 47/26

(57) ABSTRACT

The present disclosure relates to compositions comprising admixtures of poly (N-vinyl caprolactam)-poly (vinyl acetate)-poly (ethylene glycol) graft copolymer and an aqueous solution which includes i) a buffer comprising a weak acid and its conjugate base provided that at least one pKa of the weak acid is from 3 to 7.2 and/or ii) one or more sugar alcohols. The content of the copolymer in the admixture is from 12.5 wt-% to 15 wt-%. These compositions are pipetable and injectable at room temperature but form a gel at body temperature. The disclosure relates also to pharmaceutical formulations comprising the composition, and use of the composition as a thermoresponsive formulation for active pharmaceutical ingredients.

### FIELD

The present disclosure concerns compositions comprising a thermoresponsive polymer, in particular compositions comprising admixtures of poly (N-vinyl caprolactam)-poly (vinyl acetate)-poly (ethylene glycol) graft copolymer and aqueous buffers and/or aqueous sugar alcohols, and pharmaceutical formulations comprising the composition.

### BACKGROUND

Thermoresponsive polymers have been used extensively for several applications including food additives, pharmaceutical formulations, therapeutic delivery, cosmetics, and environmental remediation. The most known polymer to exhibit thermoresponsive behaviour in aqueous solution is poloxamer. Poloxamers are polyethyleneoxide-polypropyleneoxide-polyethyleneoxide (PEO—PPO-PEO) triblock copolymers that transition to gel from aqueous solutions at elevated temperatures. The exact gel transition is governed by the poloxamer subtype, the concentration of the polymer and the aqueous medium.

Soluplus® is a poly (N-vinyl caprolactam)-poly (vinyl acetate)-poly (ethylene glycol) graft copolymer with a molecular weight of 90-140 kg mol⁻¹ manufactured by BASF. It is used as a pharmaceutical excipient, as a matrix former for the production of solid dispersions, and as a surfactant to enhance the aqueous solubility of poorly water-soluble drugs.

It is known that aqueous solutions of Soluplus® undergo an increase in viscosity upon warming making it an attractive candidate as a thermothickening agent. However, it has been reported that the thickening requires the use of Soluplus® concentrations as high as 30 w/v-% (Salah et al, J. Appl. Polym. Sci. 2019; 136 (1): 1-9. doi: 10.1002/app.46915). Furthermore, the polymer has reported form thickened fluids rather than elastic gels (Abou-Shamat et al. Mol Syst Des Eng. 2020; 5 (9): 1538-1546. doi: 10.1039/d0me00093k). These may cause problems when producing pharmaceutical formulations comprising Soluplus® as thermoresponsive agent and one or more active pharmaceutically active ingredients (APIs).

Accordingly, there is still need for further thermosensitive compositions.

### SUMMARY

The present invention is based on the observation that the Soluplus® concentration required for obtaining desired thermothickening properties could be reduced when the polymer was admixed with certain buffer and/or sugar alcohol solutions.

Accordingly, it is an object of the present invention to provide a composition comprising an admixture of
poly (N-vinyl caprolactam)-poly (vinyl acetate)-poly (ethylene glycol) graft copolymer with a molecular weight of 90-140 kg mol⁻¹ and
an aqueous solution comprising
   a buffer comprising a weak acid and its conjugate base, wherein the weak acid comprises a pKₐ of 3-7.2 and/or
   one or more sugar alcohols,
wherein concentration of the poly (N-vinyl caprolactam)-poly (vinyl acetate)-poly (ethylene glycol) graft copolymer in the admixture is from 12.5 wt-% to 15 wt-%.

It is also an object of the present invention to provide a new pharmaceutical formulation comprising one or more active pharmaceutical ingredients (APIs) and the composition according to claim 1.

It is still an object of the present invention to provide a new use of a composition of claim 1 as a thermoresponsive formulation for an API.

A number of exemplifying and non-limiting embodiments of the invention are described in accompanied dependent claims.

Various exemplifying and non-limiting embodiments of the invention and to methods of operation, together with additional objects and advantages thereof, are best understood from the following description of specific exemplifying embodiments when read in connection with the accompanying figures.

The verbs “to comprise” and “to include” are used in this document as open limitations that neither exclude nor require the existence of also unrecited features.

The features recited in depending claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

The exemplifying and non-limiting embodiments of the invention are explained in greater detail below with reference to the accompanying figures, in which

FIG. 1 shows viscosity (top) and storage and loss moduli (bottom) of 30 wt-% Soluplus® in water as a function of temperature,

FIG. 2 shows viscosity (top) and storage and loss moduli (bottom) of 15 wt-% Soluplus® in 0.1 M citrate buffer (pH 6.5) as a function of temperature,

FIG. 3 shows viscosity (top) and storage and loss moduli (bottom) of 15 wt-% Soluplus® in 0.1 M Tris-HCl buffer (pH 7.3) as a function of temperature,

FIG. 4 shows viscosity (top) and storage and loss moduli (bottom) of a formulation comprising 10 wt-% piroxicam and 90 wt-% 0.1 M acetate buffer (pH 5.9) comprising 15 wt-% Soluplus® as a function of temperature,

FIG. 5 shows viscosity (top) and storage and loss moduli (bottom) of a formulation comprising 50 wt-% piroxicam and 50 wt-% 0.1 M acetate buffer (pH 5.9) comprising 15 wt-% Soluplus® as a function of temperature,

FIG. 6 shows viscosity (top) and storage and loss moduli (bottom) of a formulation comprising 30 wt-% ezetimibe and 70 wt-% 0.1 M acetate buffer (pH 5.9) comprising 15 wt-% Soluplus® as a function of temperature, and

FIG. 7 shows viscosity (top) and storage and loss moduli (bottom) of 15 wt-% Soluplus® in 0.1 M acetate buffer (pH 5.9) as a function of temperature.

### DESCRIPTION

The specific examples provided in the description given below should not be construed as limiting the scope and/or the applicability of the appended claims. Lists and groups of examples provided in the description given below are not exhaustive unless otherwise explicitly stated.

According to one aspect the present disclosure concerns a composition comprising an admixture of poly (N-vinyl caprolactam)-poly (vinyl acetate)-poly (ethylene glycol) graft copolymer with a molecular weight of 90-140 kg mol⁻¹ and an aqueous solution comprising
a buffer comprising a weak acid and its conjugate base, wherein the weak acid comprises a pKₐ of 3-7.2 and/or
one or more sugar alcohols.

Concentration of the poly (N-vinyl caprolactam)-poly (vinyl acetate)-poly (ethylene glycol) graft copolymer in the admixture is from 12.5 wt-% to 15 wt-%.

The buffer suitable for the present technology comprise acids, preferably carboxylic acids which have one or more pKₐ values between 3 and 7.2, preferably between 3 and 5. Exemplary carboxylic acids are citric acid (pKₐ 3.13, 4.76, and 6.40), formic acid (pKₐ 3.75); benzoic acid (pKₐ 4.20), and acetic acid (pKₐ 4.75), preferably citric acid and acetic acid. The buffer concentration is preferably between 0.01 M and 1 M, more preferably between 0.05 M and 0.15 M such as 0.1 M.

The carboxylic acid does not need to be a carboxylic acid. An exemplary acid weak acid which is not carboxylic acid suitable for the present technology is phosphoric acid (pKₐ₁=2.2; pKₐ₃=7.2; pKₐ₃=12.4).

According to one embodiment the admixture comprises sugar alcohols either alone or together with the buffer. As defined herein a sugar alcohol, also called polyhydric alcohol, polyalcohol, alditol or glycitol, is an organic compound, typically derived from sugars, containing one hydroxyl group (—OH) attached to each carbon atom.

Exemplary sugar alcohols suitable for the present technology are selected from a group consisting of erythritol, threitol, arabitol, xylitol, ribitol, mannitol, sorbitol, galactitol, fucitol, iditol, inositol, volemitol, isomalt, maltitol, lactitol, maltotritol, maltotetraitol, polyglycitol and mixtures thereof, preferably erythritol, xylitol, sorbitol, mannitol, and mixtures thereof, more preferably mannitol.

When the admixture comprises one or more sugar alcohols, total concentration of the sugar alcohols is preferably 10-20 wt-%, such as 15 wt-%. According to an exemplary embodiment the aqueous solution comprises 20 wt-% mannitol. According to another exemplary embodiment the aqueous solution comprises 10 wt-% mannitol.

According to another aspect the present technology concerns a pharmaceutical formulation comprising the composition described above and one or more APIs. According to an embodiment the pharmaceutical formulation comprises from 0.5 wt-% to 50 wt-% API, such as 10 wt-%, 30 wt-% or 50 wt-% API. Exemplary APIs suitable for the present technology are piroxicam and ezetimibe.

According to an exemplary embodiment the pharmaceutical formulation is prepared by admixing the composition comprising the admixture of poly (N-vinyl caprolactam)-poly (vinyl acetate)-poly (ethylene glycol) graft copolymer with a molecular weight of 90-140 kg mol⁻¹ and the aqueous solution comprising a buffer comprising a weak acid and its conjugate base, wherein the weak acid comprises a pKₐ of 3-7.2 and/or one or more sugar alcohols, wherein concentration of the poly (N-vinyl caprolactam)-poly (vinyl acetate)-poly (ethylene glycol) graft copolymer in the admixture is from 12.5 wt-% to 15 wt-% and desired amount of one or more APIs.

It is obvious for a skilled person that the preparation of the pharmaceutical formulation is not restricted to the example shown above. For example, it is possible to first admix Soluplus® and API to form an admixture, and then dilute the admixture with the aqueous solution.

According to still another aspect the present technology concerns use of a composition comprising an admixture of poly (N-vinyl caprolactam)-poly (vinyl acetate)-poly (ethylene glycol) graft copolymer with a molecular weight of 90-140 kg mol⁻¹ and an aqueous solution comprising
a buffer comprising a weak acid and its conjugate base, wherein pKₐ(s) of the weak acid is/are from 3 to 7.2, and/or
one or more sugar alcohols,
wherein concentration of the poly (N-vinyl caprolactam)-poly (vinyl acetate)-poly (ethylene glycol) graft copolymer in the admixture is from 12.5 wt-% to 15 wt-% as a thermoresponsive agent in particular for a pharmaceutical formulation.

### Experimental

### pH Measurement

The pH of the buffers prepared was determined with a Mettler Toledo PH meter.

### Temperature Dependent Flow Behavior

Flow properties of the samples prepared were evaluated at 4° C., 21° C., and 37° C. after at least 5 minutes of equilibration time.

### Rheological Characterization

For the rheological characterization of the solutions/gels an Anton-Paar MCR 302 rheometer (Anton Paar GmbH, Graz, Austria) was used. Throughout the study PP20 stainless steel 20 mm diameter parallel plate geometry (Anton Paar GmbH, Graz, Austria) and 1 mm gap was used. To prevent evaporation of sample and to regulate the temperature throughout the measurements, an evaporation blocking system equipped with a Peltier unit was used. Temperature ramp was performed as shown on the figures. Rotational tests (flow curves and viscosity curves) were performed by controlling the shear rate typically from 0.01 to 1000 s⁻¹, and measuring torque, shear viscosity and shear stress. Viscosity measurements were performed at a constant shear rate of s⁻¹ during the temperature ramp.

### Results

Admixtures comprising Soluplus® and various aqueous solutions prepared are listed in table 1. Also, their gel formation ability at body temperature is disclosed.

TABLE 1

| Soluplus wt-% | Aqueous solution | pH | Gel^{a} |
|---|---|---|---|
| 30.0 | water | n/a | yes |
| 15.0 | 0.1M acetate | 5.0; 5.9 | yes |
| 12.5 | 0.1M acetate | 5.9 | yes |
| 20.0 | 0.1M acetate | 5.9 | yes |
| 30.0 | 0.1M acetate | 5.9 | yes |
| 15.0 | Mannitol 150 mg/mL | n/a | yes |
| 12.5 | Mannitol 150 mg/mL | n/a | yes |
| 15.0 | 0.1M citrate | 4.5; 5.5; 6.5 | yes |
| 12.5 | 0.1M citrate | 4.5; 5.5; 6.5 | yes |
| 15.0 | 0.02M citrate | 6.2 | yes |
| 20.0 | 0.1M citrate | 5.0 | yes |
| 25.0 | 0.1M citrate | 5.0 | yes |
| 30.0 | 0.1M citrate | 5.0 | yes |
| 30.0 | 0.1M Tris | 7.0 | yes |
| 20.0 | 0.1M Tris | 7.0 | no |
| 15.0 | 0.1M Tris | 7.3 | no |
| 12.5 | 0.1M Tris | 7.3 | no |
| 12.5 | 0.1M phosphate | 7.0 | yes |
| 15.0 | 0.1M phosphate | 7.0 | yes |
| 15.0 | 0.01M HCl | 2 | no |
| 15.0 | 0.1% acetic acid | n/a | no |
| 12.5 | 0.1M borate | 9.2 | no |
| 15.0 | 0.1M borate | 9.2 | no |

^{a}Forms a gel at body temperature

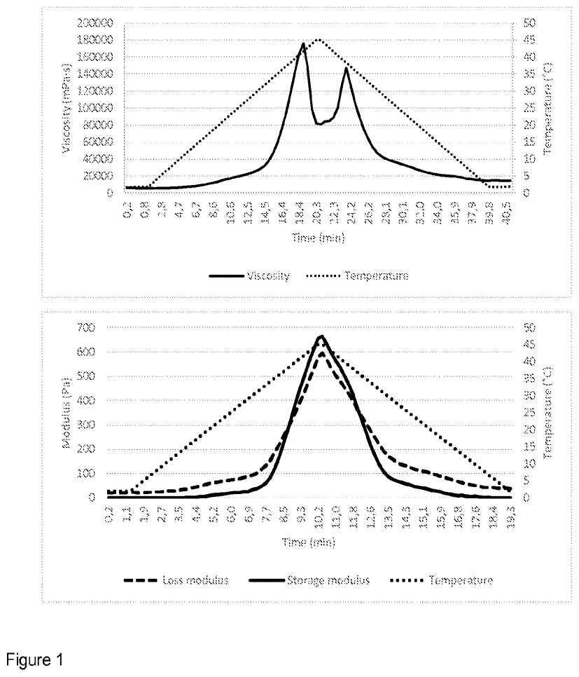

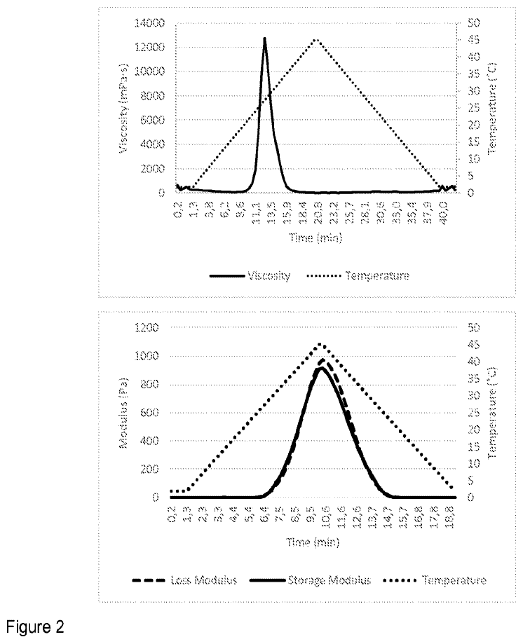

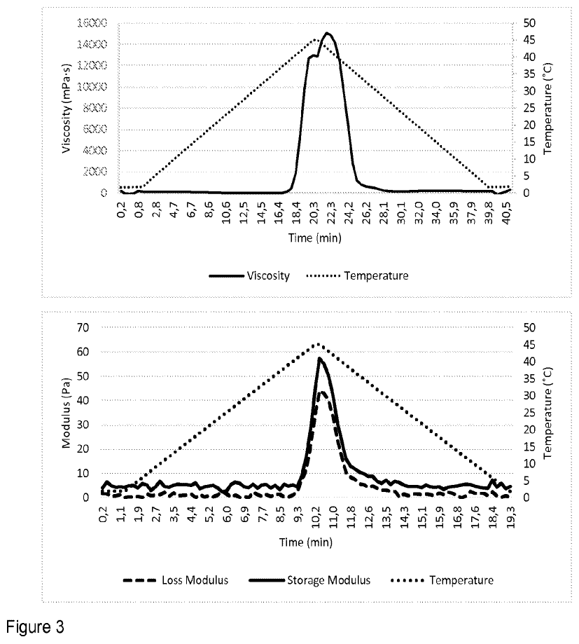

Viscosity and storage and loss moduli of 30 wt-% Soluplus® in water as a function of temperature are shown in FIG. 1. The same parameters for 15 wt-% Soluplus® in 0.1 M citrate buffer and in 0.1 M Tris-HCl buffer are shown in FIGS. 2 and 3, respectively. The results shown in table 1 and in the figures can be summarized as follows
The 30 wt-% Soluplus® in water cannot be handled by conventional pipettes because of the high viscosity.
The 30 wt-% Soluplus® in water behaves as a viscous liquid at fridge and room temperatures. The viscosity is 5 000-15 000 m·Pas, i.e. four orders of magnitude above the viscosity of room temperature water, appr. 1 m·Pas, and the loss modulus exceeds the storage modulus.
Around 32-33° C. the viscosity of 30 wt-% Soluplus® starts to increase sharply, and around 37-38° C. the storage modulus exceeds the loss modulus indicating more solid-like behaviour.
30 wt-% Soluplus® at 40-45° C. exhibits decreased viscosity.
An admixture comprising Tris-HCl was able to form a gel only when Soluplus® concentration was as high as 30 wt-%.
15 wt-% Soluplus in aqueous acetic acid or hydrochloric acid did not form a gel at body temperature.
12.5 wt-% and 15 wt-% Soluplus® in borate buffer did not form a gel at body temperature.
By replacing the pure water medium to e.g. citrate buffer, phosphate buffer or acetate buffer, the Soluplus® amount required for gelling at body temperature can be reduced down to 12.5 wt-%. These solutions are at room much less viscous and easier to handle and can easily be pipetted or injected. However, when the buffer concentration exceeded 15 wt-%, the mixture could not be handed by conventional pipettes at room temperature.

Accordingly, an advantage of using specific buffers with lower Soluplus® content is the lower viscosity that facilitates handling with pipettes whilst maintaining the solution to gel transition at or below body temperature.

### Pharmaceutical Formulations

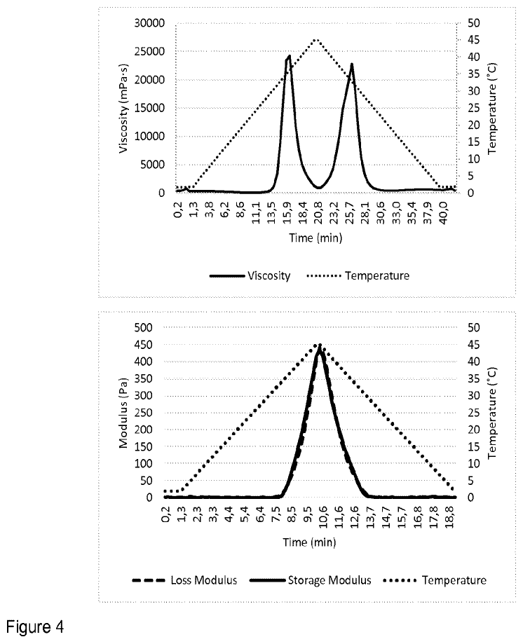

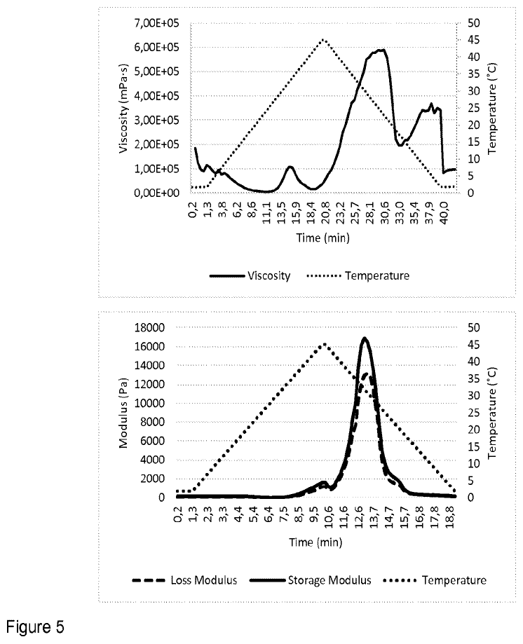

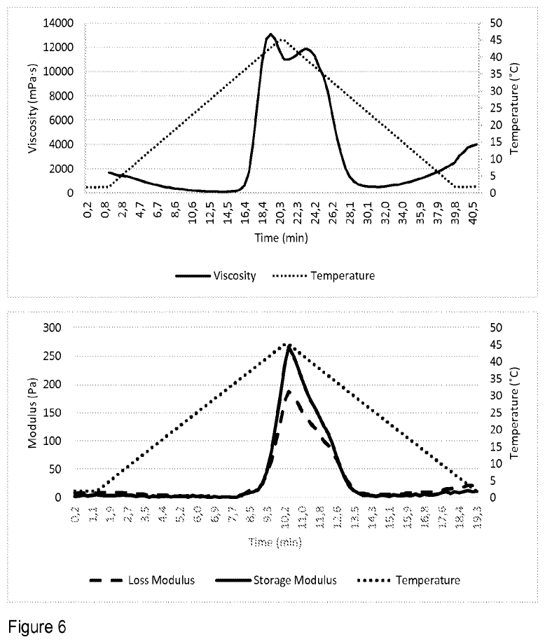

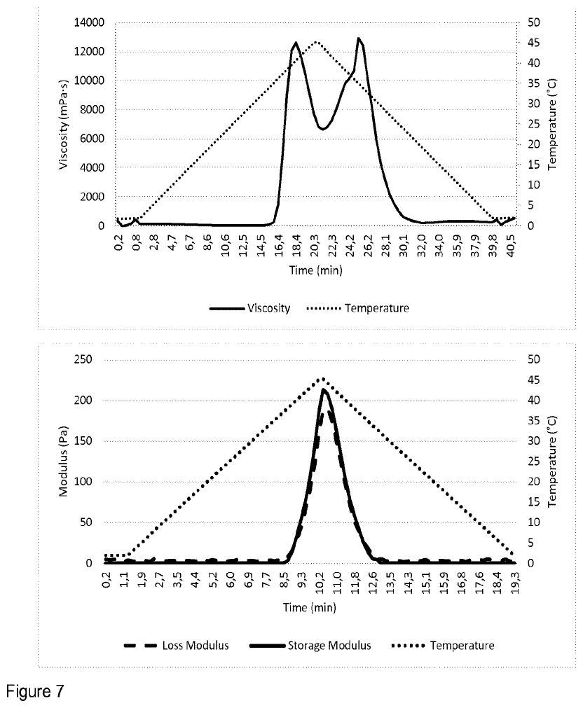

Pharmaceutical formulations were prepared by pipetting at room temperature desired amount of 0.1 M acetate buffer (pH 5.9) comprising 15 wt-% Soluplus® to known amount of API (piroxicam or ezetimibe) followed by homogenization. The formulations prepared are listed in table 2. Viscosity and storage and loss moduli of formulations comprising 10 wt-% and 50 wt-% piroxicam in 0.1 M acetate buffer (pH 5.9) comprising 15 wt-% Soluplus® as a function of temperature are shown in FIGS. 4 and 5. The corresponding data for a formulation comprising 30 wt-% ezetimibe is shown in FIG. 6. For comparison rheology data of the blank 15 wt-% Soluplus® in acetate is shown on FIG. 7.

TABLE 2

| | | | Soluplus ® in the |
|---|---|---|---|
| | API, | Composition comprising | pharmaceutical |
| API | wt-% | Soluplus ®, wt-% | formulation, wt-% |
| piroxicam | 10 | 90 | 13.5 |
| piroxicam | 30 | 70 | 10.5 |
| piroxicam | 50 | 50 | 7.5 |
| ezetimibe | 30 | 70 | 10.5 |

As seen from the figures, the formulations formed a gel at elevated temperature. The addition of an API to the system increased viscosity and storage modulus. At 50 wt-% piroxicam loading the storage modulus exceeds 15 000 Pa, indicating high resistance to deformation and high mechanical rigidity.

1. A composition comprising an admixture of poly (N-vinyl caprolactam)-poly (vinyl acetate)-poly (ethylene glycol) graft copolymer with a molecular weight of 90-140 kg mol⁻¹ and an aqueous solution characterized in that the aqueous solution comprises
a buffer comprising a weak acid and its conjugate base, wherein at least one pKₐ of the weak acid is from 3 to 7.2, and/or
one or more sugar alcohols
and in that concentration of the poly (N-vinyl caprolactam)-poly (vinyl acetate)-poly (ethylene glycol) graft copolymer in the admixture is from 12.5 wt-% to 15 wt-%.

2. The composition according to claim 1 wherein the weak acid is phosphoric acid.

3. The composition according to claim 1 wherein the weak acid is a carboxylic acid.

4. The composition according to claim 3, wherein the carboxylic acid is selected from a group consisting of citric acid, formic acid, benzoic acid, and acetic acid, preferably citric acid, and acetic acid.

5. The composition according to claim 1 wherein pH of the aqueous solution is 5-7.

6. The composition according to claim 1 wherein concentration of the buffer is from 0.01 M to 1 M, preferably from 0.05 M to 0.15 M, more preferably 0.1 M.

7. The composition according to claim 1 wherein the one or more sugar alcohols are selected from a group consisting of erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol and mixtures thereof, preferably erythritol, xylitol, sorbitol, mannitol, and mixtures thereof, more preferably mannitol.

8. The composition according to claim 1 comprising 10-20 wt-% one or more sugar alcohols.

9. A pharmaceutical formulation comprising the composition according to claim 1 and one or more active pharmaceutical ingredients (APIs).

10. The pharmaceutical formulation according to claim 9 comprising from 0.5 wt-% to 50 wt-% of the one or more APIs.

11. Use of the composition according to claim 1 as a thermoresponsive formulation for one or more APIs.
